(19) United States
(12) Patent Application Publication
(10) Pub. No.: US 2023/0165991 A1
(43) Pub. Date: Jun. 1, 2023

(54) APPARATUS FOR DISINFECTING A ROBOT

(71) Applicant: BIODECH, Inc., Suwon-si (KR)

(72) Inventors: Jinwoo KIM, Suwon-si (KR); Kyunga Ryu, Uiwang-si (KR)

(21) Appl. No.: 18/056,852

(22) Filed: Nov. 18, 2022

(30) Foreign Application Priority Data
Nov. 26, 2021 (KR) 10-2021-0165238

(51) Int. Cl. A61L 2/24; A61L 2/22; A61L 2/18; A61L 2/10; B65F 1/14; G05B 19/4155

(52) U.S. Cl. CPC A61L 2/24; A61L 2/22; A61L 2/18; A61L 2/10; B65F 1/141; G05B 19/4155; A61L 2202/14; A61L 2202/15; A61L 2202/11; A61L 2202/16; B65F 2210/168; G05B 2219/50391

(57) ABSTRACT

The present invention provides an apparatus for disinfecting a robot, and more particularly, an apparatus for disinfecting a robot for preventing secondary infection by disinfecting bodies of robot devices such as an autonomous mobile robot that carries out delivery or quarantine work in a medical field or a multi-use facility.

The apparatus for disinfecting a robot according to an embodiment of the present invention includes a disinfection apparatus main body formed so that a robot is accommodated in an inner space thereof, a disinfection unit installed on an inner surface of the disinfection apparatus main body to disinfect an outer surface of the robot, a sensor formed on an inner upper surface of the disinfection apparatus main body to detect a position of the robot, and a controller installed in the disinfection apparatus main body to control operations of the disinfection unit and the sensor. In addition, the apparatus for disinfecting a robot may be configured as a set, paired with another device, including an autonomous mobile robot equipped with a container for medical waste collection.

### CROSS REFERENCE TO RELATED APPLICATIONS

This application claims the priority benefit of Korean Patent Application No. 10-2021-0165238 filed on Nov. 26, 2021, the entirety of which is incorporated by reference herein.

### Acknowledgement Information

The present invention has been completed on the basis of the results generated by the development of non-face-to-face automation smart treatment technology, using self-driving robots of infectious medical wastes in high-risk medical environment, as part of a project for the development of infectious medical control, managed by the Korea Environmental Industry and Technology Institute, an affiliate of the Ministry of Environment, wherein the result was obtained by BIODECH, Inc., Ajou University Industry-Academic Cooperation Foundation, and Kongtech Co., Ltd., who had implemented the project since Apr. 1, 2021.

### BACKGROUND OF THE INVENTION

### 1. Field of the Invention

The present invention relates to an apparatus for disinfecting a robot, and more particularly, to an apparatus for disinfecting a robot, which disinfects surfaces of autonomous mobile robots or such robot devices when there is a risk of infection with a pathogen, during the process of work performed by autonomous mobile robots, such as delivery or quarantine, in order to prevent the possibility of secondary infection in spaces and human bodies caused by the spread of infectious pathogens while robots move around in the facilities. This disinfection apparatus may be applied to all facilities that use the autonomous mobile robots, especially medical institutions, accommodation facilities and the like.

### 2. Description of the Related Art

Unless otherwise indicated herein, the contents described in this section are not prior art to the claims of the present application, and inclusion in this section is not admitted to be the prior art.

In general, among simple repetitive tasks, cases in which a robot performs the tasks on behalf of humans in 3D (dirty, dangerous and difficult) industries are increasing.

Among them, robots that can be exposed to the risk of infection by pathogens include non-face-to-face delivery robots of infectious wards, non-face-to-face collection robots of medical waste, space sterilization robots (UV rays for disinfection, a type of spraying disinfectant, etc.) and the like. While these robots move between the infected area and the general area, there is a risk of spreading pathogens in the facility in a state of being exposed to dangerous pathogens.

In this regard, Korean Patent Registration Publication No. 10-2102784 discloses an autonomous mobile disinfection robot system. More specifically, the autonomous mobile disinfection robot system includes: an autonomous mobile disinfection robot capable of unmanned autonomous driving while spraying a humidifying or disinfecting fluid; a server configured to control the humidifying or disinfecting fluid sprayed from the robot and receive data on the autonomous driving of the robot; and a user terminal configured to receive the data transmitted from the server.

The autonomous mobile disinfection robot as described above has a risk of spreading pathogens when moving between areas for delivery, but the robot cannot disinfect pathogens by itself. In addition, the disinfection robot is a device for disinfecting air or a surface of an article, and whether or not self-disinfection can be performed may vary depending on the disinfection method. For example, in the case of a vapor type apparatus among apparatuses using a disinfectant, after the space to be disinfected is closed, the disinfectant is vaporized to make it into a saturated vapor state. For that reason, the robot disposed in the closed space are disinfected together. However, even if using the same disinfectant, in the case of the spray type robot, self-disinfection cannot be performed, such that the surface should be manually wiped or a certain self-disinfection apparatus is required additionally. In particular, in the case of a disinfection robot using an ultraviolet light source, specific objects within robots' driving regions can be disinfected, but there is a disadvantage that the robots themselves are not able to disinfect the surfaces of their bodies.

Recently, due to highly-infectious viruses such as coronavirus, the need to disinfect not only objects requiring disinfection, but also autonomous mobile robots used for delivery and disinfection work in infected areas and clean areas is becoming a primary issue.

### REFERENCES

### [Patent Document]

(Patent Document 1) Korean Patent Registration Publication No. 10-2102784 (published on Apr. 29, 2020)

### SUMMARY OF THE INVENTION

An object of the present invention is to provide an apparatus for disinfecting a robot intended to prevent secondary infection by sterilizing the autonomous mobile robot itself that transports items such as a disinfectant and performs quarantine work on behalf of humans in a place exposed to viruses.

In addition, it is not limited to the technical problems as described above, and it is obvious that another technical problem may be derived from the following description.

To achieve the above objects, according to an aspect of the present invention, there is provided an apparatus for disinfecting a robot including: a disinfection apparatus main body formed so that a robot is accommodated in an inner space thereof, a disinfection unit installed on an inner surface of the disinfection apparatus main body to disinfect an outer surface of the robot; a sensor formed on an inner upper surface of the disinfection apparatus main body to detect a position of the robot; and a controller installed in the disinfection apparatus main body to control operations of the disinfection unit and the sensor.

According to a preferred feature of the present invention, the disinfection unit may include: a pair of shafts coupled to connection portions extending forward from the inner surface of the disinfection apparatus main body; a disinfection member detachably installed on the pair of shafts and configured to disinfect the outer surface of the robot; a vertical transfer guide configured to raise and lower the connection portions; at least one disinfecting fluid spray nozzle installed on the inner surface of the disinfection apparatus main body to spray a disinfecting fluid toward the robot or the disinfection member.

According to a preferred feature of the present invention, the apparatus may further include: a disinfecting fluid storage tank installed inside the disinfection apparatus main body, and configured to store and supply the disinfecting fluid to the disinfecting fluid spray nozzle; and a disinfecting fluid pump configured to deliver the disinfecting fluid, which is stored in the disinfecting fluid storage tank, to the disinfecting fluid spray nozzle.

According to a preferred feature of the present invention, the apparatus may further include: a sterilization device installed on the inner upper surface or a lower surface of the disinfection apparatus main body to sterilize the robot using an ultraviolet light source.

According to a preferred feature of the present invention, the apparatus may further include: an air spray device installed on the inner surface of the disinfection apparatus main body to spray air toward the robot to remove contaminated particles on the surface of the robot; and a particle collection device configured to collect the particles removed from the surface of the robot by the air spray device.

According to a preferred feature of the present invention, the controller may move the pair of shafts, on which the disinfection member is installed, to all directions around the robot to disinfect the outer surface of the robot.

According to a preferred feature of the present invention, after performing disinfection on the surface of the robot by self-rotation of robot, which is in surface contact with the disinfection member or by a motion of the pair of shafts on which the disinfection member is installed, the controller may rotate the pair of shafts to position an unused disinfection member adjacent to the robot.

According to a preferred feature of the present invention, the apparatus may further include a rotation plate installed on an inner lower surface of the disinfection apparatus main body to rotate the robot detected by the sensor about the axis of rotation in a circumferential direction.

According to a preferred feature of the present invention, the apparatus may further include a disinfection member collection device configured to collect the disinfection member used in the disinfection unit.

According to a preferred feature of the present invention, the robot may be a medical waste collection robot having a collection container configured to collect and contain medical waste, and a drive mechanism that helps the collection container to function autonomous driving, or may be a medical assistant robot including a function of transporting medical equipment or an assistive mechanism for a patient in a medical facility.

According to a preferred feature of the present invention, the collection container of the collection robot may be formed as a cylindrical container configured to contain various medical wastes, and a sterilizer which is operated by receiving a power may be installed and used in the container.

According to a preferred feature of the present invention, the drive mechanism that enables the collection container to autonomously drive may include: a body disposed below the collection container and formed to match a size and a shape of the collection container; a drive motor installed inside the body to generate a driving force; a controller configured to supply a power to the drive motor and control an operation thereof, a drive wheel driven by the power received from the drive motor; and driving wheels configured to support the body moving by the driving force of the drive wheel.

According to a preferred feature of the present invention, the drive mechanism and the collection container may be connected to a drive shaft of the drive motor through a support bearing disposed on an upper edge of the body of the drive mechanism, so that the collection container is freely rotated with respect to the drive mechanism by the driving force received from the drive motor.

According to a preferred feature of the present invention, the medical waste collection robot or the medical assistant robot may include an electrical signal transceiver configured to transmit and receive an electrical signal to perform communication therewith, respectively.

The present invention has the following advantages. According to the present invention, secondary infection and the spread of pathogens may be prevented through self-disinfection of robots which transport medical waste and perform quarantine work on behalf of humans at the place exposed to infectious pathogens such as viruses.

In addition, according to the present invention, it is possible to disinfect and sterilize the entire robot through a simple mechanical configuration.

In the apparatus of the present invention designed as the above-described configuration, when putting medical waste that has a risk of being infected with viruses or various germs into the collection container of the medical waste collection robot in a negative pressure isolation ward, etc., the collection robot first enters the disinfection apparatus before it comes out of the isolation ward. Thereafter, the disinfecting fluid provided in the disinfection apparatus is sprayed onto the collection container to sterilize, or the surface of the collection container is directly wiped with an adhesive cloth moistened with disinfecting fluid for sterilization, or by irradiating the robot with light by an electric sterilizer to sterilize, such that various germs or viruses attached to the collection container are disinfected. And finally, the robot comes out of the hospital room.

As described above, by sterilizing the medical waste collection robot using the apparatus according to the present invention, it is possible to prevent an occurrence of problems such as the spread of viruses that may be contaminated in the isolation ward to an outside by a medical waste transport robot.

Further, in the present invention, the robot is disinfected with a light source for regular operation, chemical spray, and/or a type of wiping for washing the surface of robot and a sterilization cycle with hydrogen peroxide vapor for sterilization of entire interiors, etc., such that it is possible to carry out one or more of sterilization processes for each disinfection level.

The effects of the present invention are not limited to the above advantages, and those skilled in the art should understand that it may include all effects that can be deduced from the detailed description of the present invention or the configurations of the invention described in the claims.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of an apparatus for disinfecting a robot according to an embodiment of the present invention;

FIG. 2 is a detailed view of the apparatus for disinfecting a robot according to an embodiment of the present invention;

FIG. 3 is perspective views illustrating a state in which a robot is disinfected while rotating in the apparatus for disinfecting a robot according to an embodiment of the present invention;

FIG. 4 is perspective views illustrating an operating state of a disinfection member and spray nozzles in the apparatus for disinfecting a robot according to an embodiment of the present invention;

FIG. 5 is a perspective view in a state in which front and both sides are closed in the apparatus for disinfecting a robot according to an embodiment of the present invention;

FIG. 6 is a side cross-sectional view illustrating operating states of an air spray device and a particle collection device in the apparatus for disinfecting a robot according to an embodiment of the present invention;

FIG. 7 is a plan view illustrating operating states of the air spray device and the particle collection device in the apparatus for disinfecting a robot according to an embodiment of the present invention;

FIGS. 8A-8C are views illustrating an example of the configuration of a medical waste collection robot that can be included in the device according to the invention;

FIG. 8A is a plan view illustrating the medical waste collection robot of the apparatus according to the present invention;

FIG. 8B is a front view illustrating the medical waste collection robot of the apparatus according to the present invention; and

FIG. 8C is a bottom view illustrating the medical waste collection robot of the apparatus according to the present invention.

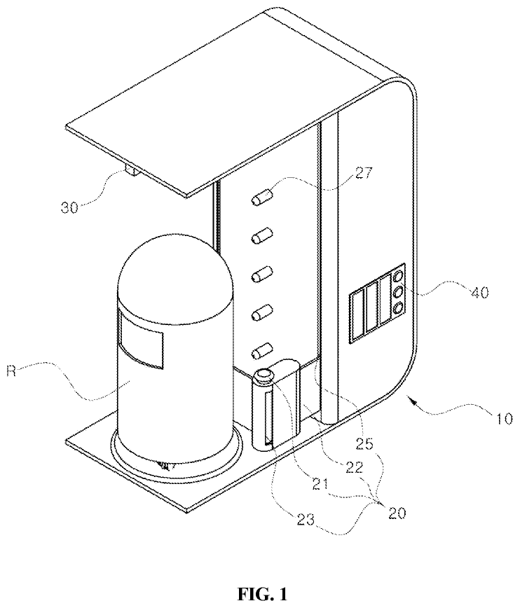

### DETAILED DESCRIPTION OF THE INVENTION

Hereinafter, configurations, operations, and effects of an apparatus for disinfecting a robot according to preferred embodiments will be described with reference to the accompanying drawings. For reference, in the drawings described below, each component is omitted or schematically illustrated for convenience and clarity, and the size of each component does not reflect an actual size. In addition, the same reference numerals are denoted to the same components throughout the specification while reference numerals for the same components in the individual drawing are omitted.

The apparatus for disinfecting a robot according to an embodiment of the present invention includes: a disinfection apparatus main body 10 formed so that a robot R can be accommodated in an inner space thereof, a disinfection unit 20 installed on an inner surface of the disinfection apparatus main body 10 to disinfect an outer surface of the robot R; a sensor 30 formed on an inner upper surface of the disinfection apparatus main body 10 to detect a position of the robot R; and a controller 40 installed in the disinfection apparatus main body 10 to control operations of the disinfection unit 20 and the sensor 30.

The robot R disclosed in the present invention may be a robot capable of autonomously driving. In addition, the inventive disinfection apparatus may be applied to a robot which is installed in a loading dock or a ward to perform various quarantine tasks, such as collecting medical waste on behalf of humans or delivering items to a dangerous infected area. For example, the inventive disinfection apparatus may be applied to two types including: a robot self-disinfection apparatus which is installed inside or near an entrance of the ward in a moving path where the robot collects medical waste from the infection control special control area (negative pressure isolation ward, operating room, intensive care unit, clean room, etc.), comes out therefrom, and moves to the loading dock through the ward, so as to prevent the spread of pathogens and secondary infection in a hospital; and a robot self-disinfection apparatus which is installed near the entrance of the hospital so as to, after the robot completes the delivery of medical waste to the storage warehouse at the loading dock, disinfect and sterilize infectious pathogens before entering the hospital again.

The robot self-disinfection apparatus may be used for the purpose of sterilizing the surfaces of robots that have performed quarantine work or devices that have been equipped in areas exposed to infectious pathogens. For this disinfection work, a disinfection cycle may be operated in conjunction with the robot or manually. Further, for easy use in various places, wheels can be mounted on the bottom of the disinfection apparatus to improve mobility. The present invention discloses an apparatus for disinfecting and sterilizing the surfaces of various robots that perform tasks such as collection, delivery, monitoring, and quarantine of items on behalf of humans in the areas exposed to infectious pathogens.

First, the inventive disinfection apparatus may include the disinfection apparatus main body 10 formed so that the robot R can be accommodated in the inner space thereof. The disinfection apparatus main body 10 is configured to have a height and a width capable of accommodating an entire volume of the robot R, and preferably, may include an open entrance space formed on one side thereof to facilitate entry to and exit from the inner surface during movement of the robot R. The disinfection for the robot R is performed on the closed upper and lower surfaces and sides based on the open entrance space.

The disinfection apparatus main body 10 may further include a disinfection unit 20 installed on an inner surface thereof to disinfect the outer surface of the robot. The disinfection unit 20 is a component intended to disinfect the robot R, and more precisely, the outer surface of the robot R. Disinfection may be performed in such a way that the disinfection unit 20 installed on the inner surface of the disinfection apparatus main body 10 wipes the outer surface of the robot R with a disinfecting fluid contained therein.

The inventive disinfection apparatus includes the sensor 30 formed on the inner upper surface of the disinfection apparatus main body 10 to detect a position of the robot R, and the controller 40 installed in the disinfection apparatus main body 10 to control operations of the disinfection unit 20 and the sensor 30. The sensor 30 is a position detection sensor for determining whether the robot R is positioned in the right location where the disinfection is performed in the disinfection apparatus main body 10. If the robot R is positioned at a predetermined location while autonomously driving, the sensor 30 detects this state and transmits a signal indicating that the robot R is detected from the sensor 30 to the controller 40, and when receiving the signal, the controller 40 operates the disinfection unit 20 to perform the disinfection of the robot R.

Herein, the controller 40 communicates with an external control center server. Therefore, the control center may monitor and control an entire process of disinfecting the robot R, and an administrator may check the disinfection process of the robot R in real time through a mobile terminal communicated with the control center server, and a time when disinfection starts and/or ends may also be checked through an alarm.

According to a preferred feature of the present invention, the disinfection unit 20 may include: a pair of shafts 21 coupled to connection portions 22 extending forward from the inner surface of the disinfection apparatus main body 10; a disinfection member 23 detachably installed on the pair of shafts 21 and configured to disinfect the outer surface of the robot; a vertical transfer guide 25 configured to raise and lower the connection portions 22; and a disinfecting fluid spray nozzle 27 installed on the inner surface of the disinfection apparatus main body 10 to spray the disinfecting fluid toward the robot R or the disinfection member 23.

As an embodiment for disinfecting the robot R through the disinfection unit 20, the present invention discloses a configuration in which disinfection is performed while wiping the outer surface of the robot R by a wiping-type disinfection member 23. First, these components may be installed in such a way that the connection portions 22 extending in a direction in which the robot R is positioned are formed on the inner surface of the disinfection apparatus main body 10, and the pair of shafts 21 are detachably coupled to the connection portions 22. The pair of shafts 21 extend to a vertical direction and have a shaft type configuration in which the shafts are installed to be spaced apart from each other by a length greater than a diameter of the robot R The disinfection member 23 to be described below may be wound and fixed to the shafts 21.

Next, the disinfection unit 20 includes the disinfection member 23 installed on the pair of shafts 21 to disinfect the outer surface of the robot R in a wiping manner. The disinfection member 23 may be a roll-type tissue which is detachably installed on the shafts 21, but it is not limited thereto. The disinfection member 23 may be formed of any material and have any configuration as long as it is a flexible material that can wipe the outer surface of the robot R in the wiping manner, such as cloth, fabric, or non-woven fabric. Herein, when the pair of shafts 21 rotate in the same direction while the disinfection member 23 is installed on one of the pair of shafts 21, the disinfection member 23 is transferred from one side of the shaft 21a to the other side of the shaft 21b. In this process, the disinfection member 23 located between the pair of shafts 21 and containing the disinfecting fluid comes in surface contact with the outer surface of the robot R, and in this state, disinfection of the outer surface is performed while the robot R rotates about an axis thereof

According to a preferred feature of the present invention, the inventive disinfection apparatus may further include a rotation plate 60 installed on an inner lower surface of the disinfection apparatus main body 10 to rotate the robot R detected by the sensor 30 about the axis of rotation in a circumferential direction.

The axial rotation of the robot R may be performed by itself inside the robot R. Alternatively, the rotation plate 60, on which the robot R is positioned, makes the rotation of the robot R by its axial rotation. Therefore, as the robot R rotates, disinfection is performed while the outer surface of the robot R is wiped by the disinfection member 23 located between the pair of shafts 21. As the shafts 21 rotate, the used disinfection member 23 is transferred from one shaft 21a to the other shaft 21b and wound thereon. When the disinfection member 23 is completely used, it may be removed from the shaft 21 and disposed.

Next, the disinfection unit 20 may include the vertical transfer guide 25 for raising and lowering the connection portions 22. The vertical transfer guide 25 is a component for raising and lowering the connection portions 22, and more precisely, the pair of shafts 21 coupled to the connection portions 22 and the disinfection member 23, and may be configured as a transfer device such as an LM guide installed on one side of the disinfection apparatus main body 10 to vertically move the connection portions 22. As the outer surface of the robot R is wiped in the wiping manner over the entire length of the robot R in the vertical direction by the disinfection member 23 vertically moved through the vertical transfer guide 25, the disinfection may be performed. In this case, as the height of the disinfection member 23 is changed step by step, and the robot R rotates by itself or is rotated by the rotation plate 60, disinfection of all outer surfaces of the robot R can be performed.

The disinfection apparatus main body 10 may include a drying device installed on the inner surface thereof, which dries the outer surface by spraying air toward the robot R, after disinfection of all outer surfaces of the robot R is completed as described above.

In addition, the disinfection unit 20 may include the disinfecting fluid spray nozzles 27 for spraying the disinfecting fluid toward the disinfection member 23. The disinfection member 23 may be a tissue containing the disinfecting fluid. Further, in the embodiment disclosed in the present invention, it may be configured in such a way that the disinfecting fluid is sprayed through the disinfecting fluid spray nozzles 27 for spraying the disinfecting fluid toward the disinfection member 23, and the disinfection member 23 absorbing the disinfecting fluid comes into contact with the robot R to disinfect the same. The disinfecting fluid spray nozzles 27 may be installed at positions corresponding to the entire length of the vertical transfer guide 25 in order to spray the disinfecting fluid at a location where the vertically moving disinfection member 23 is positioned in the vertical direction, and may be arranged in various ways according to the shape or size of the robot R, etc. Therefore, it may be configured so that the disinfection member 23 and the disinfecting liquid spray nozzles 27 for spraying the disinfecting fluid toward the disinfection member 23 can be moved in all directions.

According to a preferred feature of the present invention, the disinfection unit 20 may further include: a disinfecting fluid storage tank 11 installed inside the disinfection apparatus main body 10, and configured to store and supply the disinfecting fluid toward the disinfecting fluid spray nozzles 27, and a disinfecting fluid pump 13 configured to deliver the disinfecting fluid stored in the disinfecting fluid storage tank 11 to the disinfecting fluid spray nozzles 27.

The disinfecting fluid is stored in a certain amount in the disinfecting fluid storage tank 11 formed inside the disinfection apparatus main body 10, and when the controller 40 operates the disinfection unit 20, the disinfecting fluid pump 13 starts to operate and pump the disinfecting fluid from the disinfecting fluid storage tank 11 to the nozzles, such that the disinfecting fluid can be sprayed onto the disinfection member 23.

According to a preferred embodiment of the present invention, as a method of spraying the disinfecting fluid, various methods such as a liquid type, an aerosol type, or a vapor type spraying, etc. may be employed. In this case, it is possible to enable effective spray by using an appropriate nozzle according to a type of spraying. This spray type may vary depending on the type of disinfecting fluid.

According to the present invention, such a disinfecting fluid spray nozzle may be determined according to the spray type, and these types may be used in combination of two or more thereof. Therefore, the disinfecting fluid spray nozzle of the present invention may include one or more spray nozzle(s) selected from liquid type, aerosol type, and vapor type nozzles.

According to a preferred feature of the present invention, the disinfection apparatus of the present invention may further include a sterilization device 50 installed on the inner upper surface or lower surface of the disinfection apparatus main body 10 to sterilize the robot using an ultraviolet light source.

The sterilization device 50 using the ultraviolet light source sprays the disinfecting fluid and enables sterilization and pasteurization operations to be performed using UV rays in addition to disinfection by wiping the surface in a wiping manner. When the sterilization device 50 using the ultraviolet light source is added, preferably, the sterilization devices 50 using the ultraviolet light source are installed on the inner upper side and lower side of the disinfection apparatus main body 10 where the robot R is positioned, thereby enabling sterilization to be performed by irradiating the robot R with UV rays.

In addition, according to a preferred feature of the present invention, the inventive disinfection apparatus may further include: air spray devices 15 installed on the inner surface of the disinfection apparatus main body 10 to spray air toward the robot to remove contaminated particles on the surface of the robot; and a particle collection device 17 configured to collect the particles removed from the surface of the robot by the air spray devices 15.

The air spray device 15 is a component for removing dust and contaminated particles through an air shower function that sprays air onto the outer surface of the robot R before the disinfection operation of the robot R is performed, and supplies compressed air from an air compressor 19 installed inside the disinfection apparatus main body 10 toward the robot R to remove the contaminated particles from the outer surface of the robot.

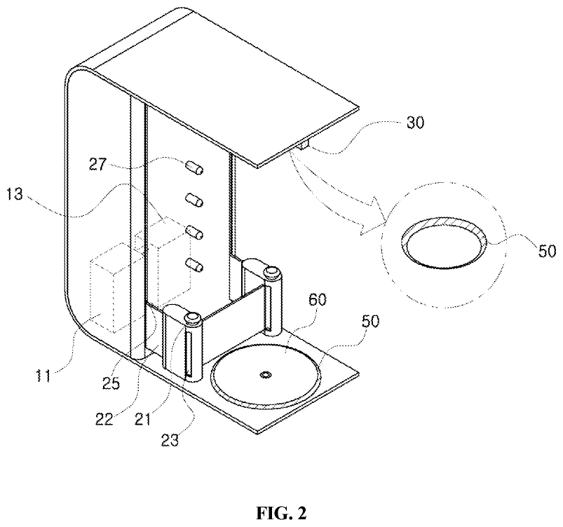

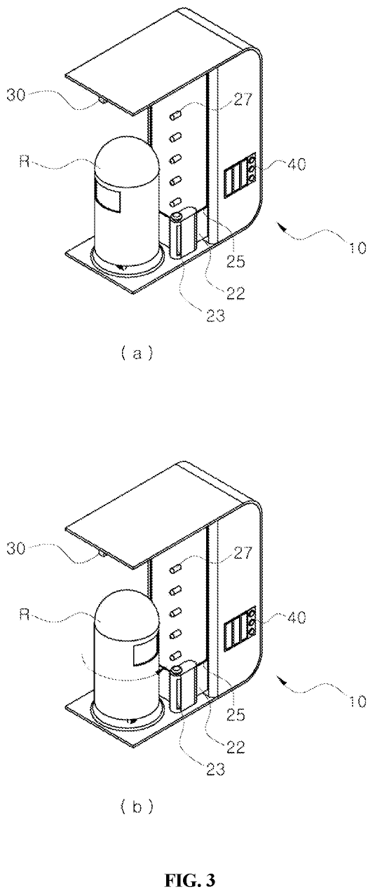

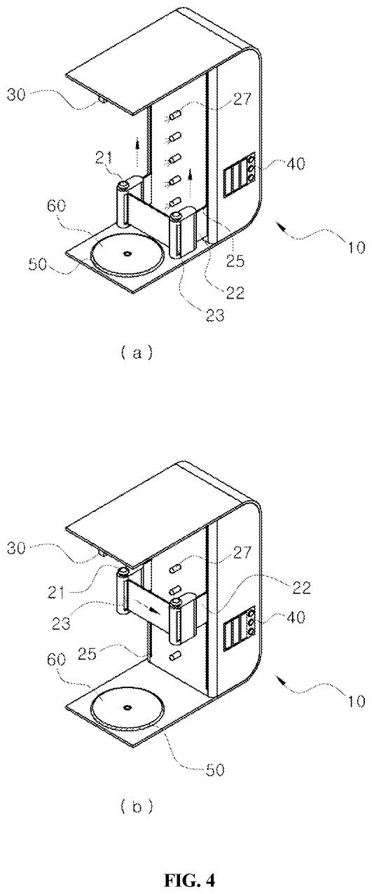

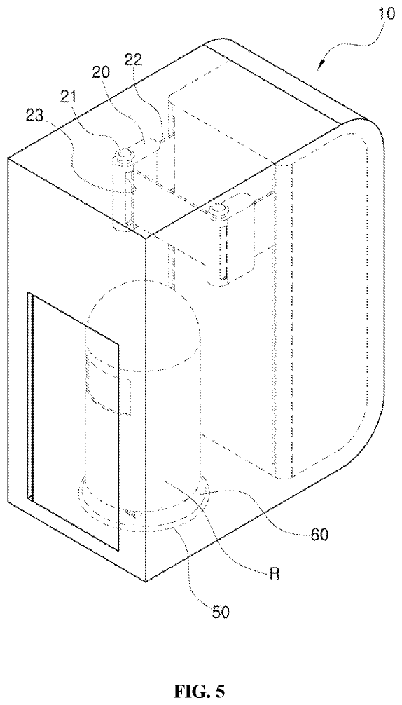

As shown in FIG. 2, it may be configured so that the air spray devices 15 are installed on top of the robot R to spray the compressed air toward the robot R to remove the contaminated particles. Preferably, it is configured so that a plurality of air spray devices 15 are installed side by side in the vertical direction on both sides of the front of the disinfection apparatus main body 10 which is made to be closed on both sides and the rear side, and to be opened and closed by an entrance door on the front side to form a completely sealed inner space. Herein, the compressed air from the air compressor 19 is used not only for the purpose of removing the contaminated particles on the outer surface of the robot R, but also may be used for the purpose of a door packing for preventing the inflow and outflow of air from minute gaps such as an entrance door in the completely sealed inner space.

The compressed air supplied from the air compressor 19 is delivered to the air spray devices 15 along the upper portion of the disinfection apparatus main body 10, to be sprayed on both sides of the front of the robot R. Therefore, the contaminated particles are removed by the compressed air, and the particles removed as described above may be collected by the particle collection device 17 formed on one inner circumferential surface of the disinfection apparatus main body 10. An air suction device capable of absorbing air transferred from the inner space may be installed at one end of the particle collection device 17. The particle collection device 17 may include a plurality of filters to filter out the contaminated particles.

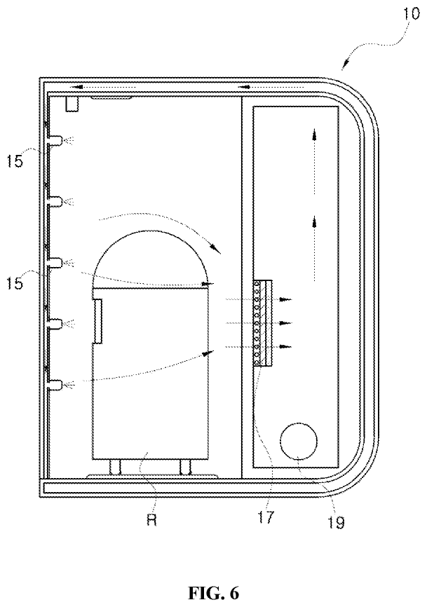

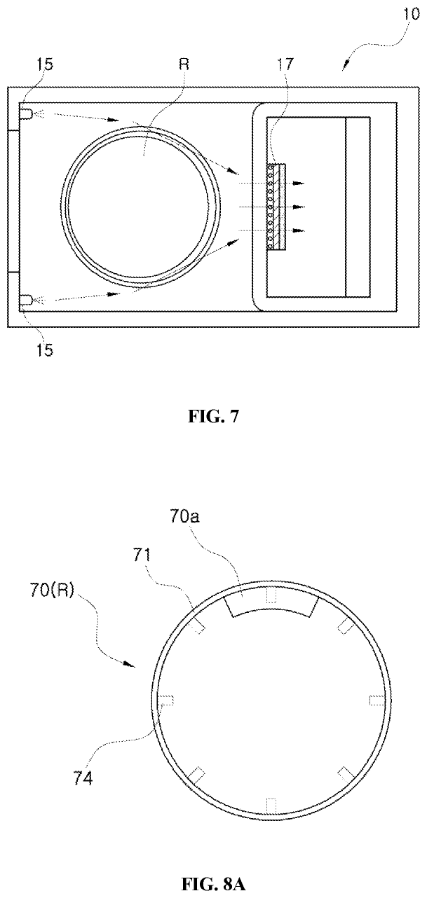

A perforated or mesh-type front panel is formed at the front end of the particle collection device 17, such that particles having large particles may be prioritized to be filtered out. In addition, a pre-filter or a HEPA filter is installed at the rear end of the front panel to additionally filter fine particles. After removing these contaminated particles, the purified air moves to the upper portion of the disinfection apparatus main body 10 along the rear surface of the disinfection apparatus main body 10 as indicated by arrows in FIG. 6 and is delivered back to the air spray devices 15, thereby facilitating air circulation in the inner space.

According to a preferred feature of the present invention, the controller 40 can move the pair of shafts 21 on which the disinfection member 23 is installed in all directions around the robot R to sterilize the outer surface of the robot R.

There is a need to configure the pair of shafts 21 on which the disinfection member 23 is installed to be movable in all directions in order to disinfect the outer surface of the robot R in the wiping manner. Therefore, the controller 40 may control the pair of shafts 21 not only to disinfect the robot while being moved in the vertical direction by the vertical transfer guide 25 about the position where the robot R is positioned, but also to move the shafts 21 in all directions, so that disinfection takes place over the entire area of the robot R.

According to a preferred feature of the present invention, after performing disinfection on the surface of the robot R as the robot R in surface contact with the disinfection member 23 rotates by itself or by the motion of the pair of shafts 21 on which the disinfection member 23 is installed, the controller 40 may rotate the pair of shafts 21 to position an unused disinfection member 23 adjacent to the robot.

When the robot R rotates by itself, is rotated by the rotation plate 60, or the pair of shafts 21 on which the disinfection member 23 is installed moves in all the directions under the control of the controller 40, the outer surface of the robot R is disinfected by the disinfection member 23 in the wiping method. When the disinfection is completed, the used tissue is removed and new unused tissue is assigned again adjacent to the robot R in order to perform the disinfection. To this end, when the robot R in surface contact with the disinfection member 23 is first disinfected, the controller 40 rotates the shafts 21 to position the unused disinfection member 23 so as to wipe the surface of the robot R. Accordingly, the possibility that the contaminated disinfection member 23 is exposed to cause an additional infection is prevented in advance, and disinfection and sterilization works are performed cleanly.

According to a preferred feature of the present invention, the disinfection apparatus of the present invention may further include a disinfection member collection device configured to collect the disinfection member 23 used in the disinfection unit 20.

There is a need to stably collect and remove the used disinfection member 23. To this end, the disinfection member 23 used in the disinfection unit 20 is wound on the other shaft 21b, and after use, all the wound used disinfection member 23 is collected by the disinfection member collection device so as to be stably disposed. Therefore, it is possible to prevent the secondary infection that may occur during the movement of the robot in advance. Furthermore, sterilization devices such as UV sterilization lamps may be installed at an entrance portion through which the disinfection member 23 flows from the disinfection member collection device, such that additional sterilization and pasteurization works may be performed on the used disinfection member.

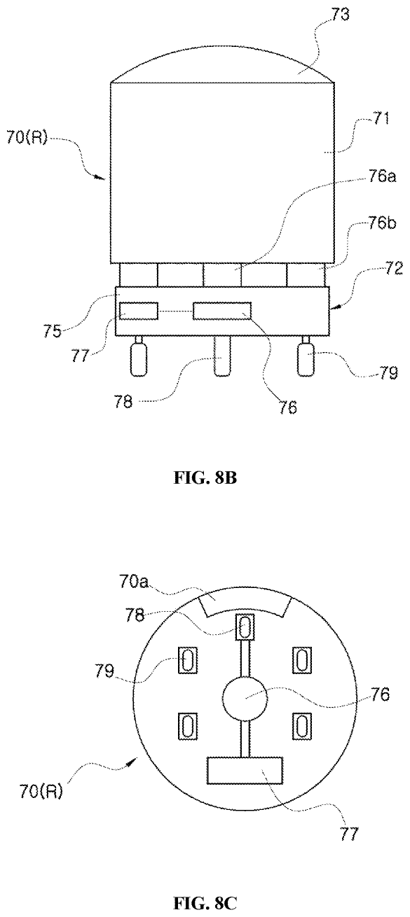

Meanwhile, according to another preferred embodiment of the present invention, as an example of the robot R used in the apparatus for disinfecting a robot, the present invention further discloses an apparatus for disinfecting a robot, which is formed as a set, paired with another device, a robot, preferably, for example, a medical waste collection robot illustrated in FIGS. 8A to 8C.

For example, the apparatus for disinfecting a robot according to another embodiment of the present invention includes: a collection container 71 capable of collecting and containing medical waste; and a medical waste collection robot 70 having a drive mechanism 72 for autonomously driving the collection container 71. That is, the apparatus for disinfecting a robot of this embodiment may be configured as a set-type disinfection apparatus formed as one set by further including the collection robot 70 having the above-described collection container 71. In particular, when configuring the apparatus for disinfecting a robot of the present invention in such the set type, the medical waste collection robot 70 and the apparatus for disinfecting a robot for disinfecting may be configured to match the shape and the size compatible with each other, and may perform communication with each other in a mutually agreed manner, such that autonomous disinfection in a manner in which processes such as movement and disinfection, etc., are controlled is possible. Further, in the case of such the set-type disinfection apparatus, since disinfection is performed while maintaining compatibility between the apparatus for disinfecting a robot and the robot R such as the collection robot 70, for example, it may be said to be a preferable form in an aspect that the possibility of disinfection in an incomplete state is eliminated. Therefore, the set-type disinfection apparatus including the collection robot is more advantageous in an aspect that an automated disinfection system can be implemented in areas requiring high sanitary conditions such as negative pressure hospital rooms, disease-contaminated areas, infected areas and the like.

According to the preferred embodiment of the present invention, as shown in detail in FIGS. 8A, 8B, and 8C, the collection robot 70 includes the collection container 71 for containing medical waste and the drive mechanism 72 for autonomously driving the collection container 71.

The collection container 71 is formed as a cylindrical container capable of containing various medical wastes, and may be provided with a lid 73 as necessary, and an ultraviolet sterilizer 74 which is operated by receiving a power may be installed and used inside the collection container 71.

In addition, as shown in FIG. 8B, the drive mechanism 72 that enables the collection container 71 to autonomously drive may include: a body 75 disposed below the collection container 71 and formed to match the size and shape of the collection container; a drive motor 76 installed inside the body 75 to generate a driving force; a controller 77 configured to supply a power to the drive motor 76 and control an operation thereof, a drive wheel 78 driven by the power received from the drive motor 76; and driving wheels 79 configured to support the body 75 moving by the driving force of the drive wheel 78.

The collection container 71 and the drive mechanism 72 have a structure connected to a drive shaft 76a of the drive motor 76 through a support bearing 76b disposed on an upper edge of the body of the drive mechanism 72. Accordingly, the collection container 71 is configured to be freely rotated with respect to the drive mechanism 72 by the driving force received from the drive motor 76.

The drive mechanism 72 having the above-described structure is configured so that the driving force of the drive motor 76 is directly transmitted to the drive wheel 78, and includes a signal transceiver 70a for the collection robot 70 configured to facilitate a communication for transmitting and receiving an electrical signal to and from the sensor 30 installed in the disinfection apparatus main body 10 of the apparatus for disinfecting a robot so as to be moved. According to the signal input and output to and from the signal transceiver 70a, the collection container 71 is configured to move in a desired direction.

Therefore, the controller 77 of the collection robot 70 moves and freely rotates the collection container 71 by the power of the drive motor 76 based on the signal detected, and positions it on the rotation plate 60 of the apparatus for disinfecting a robot. Accordingly, it is possible to disinfect the virus and bacteria attached to the surface of the collection container 71. In this case, it may be operated so that the rotation plate 60 and the collection robot 70 rotate a plurality of times with each other, or only one of them rotates for the completion of disinfection thoroughly. Further, in some cases, it may be configured so that the collection robot 70 is positioned on a fixed plate without the rotation plate 60.

When the disinfection of the collection container 71 is completed by the apparatus for disinfecting a robot as described above, the collection robot 70 may come out of the rotation plate 60 of the apparatus for disinfecting a robot or its position to complete the disinfection work.

As described above, while the collection robot collects the contaminated waste such as medical waste discharged from the negative pressure isolation ward and comes out therefrom, the apparatus according to the present invention allows the robot to be safely disinfected through the disinfection apparatus before it comes out from the ward and then exits the hospital room while moving along a route predetermined by a separate remote control or program. Therefore, it is possible to prevent an occurrence of a problem such as contamination of the surroundings due to the robot for collecting medical waste.

In addition, this apparatus for disinfecting a robot may be utilized to enable disinfection of medical waste collection robots, etc., outside the hospital room in a similar way as described above.

Further, during performing disinfection on the collection robot, one of a non-contact disinfection method, a spray method of disinfecting an article by spraying a disinfecting fluid, or a contact disinfection method of directly applying the disinfecting fluid to a subject may be selectively used as necessary.

Although preferred embodiments of the present invention have been described with reference to the accompanying drawings, the embodiments and drawings described and illustrated in the present disclosure are simply the most preferred embodiment and do not represent all the technical sprites of the present invention, and it shall be understood that various modifications and equivalents may be made to take the place of the embodiments at the time of filling the present application. Thus, it is clear that the above-described embodiments are illustrative in all aspects and do not limit the present disclosure. The scope of the present disclosure is defined by the following claims rather than by the detailed description of the embodiment. It shall be understood that all modifications and embodiments conceived from the meaning and scope of the claims and their equivalents are included in the scope of the present disclosure.

### DESCRIPTION OF REFERENCE NUMERALS

10: Disinfection apparatus main body
11: Disinfecting fluid storage tank
13: Disinfecting fluid pump
15: Air spray device
17: Particle collection device
19: Air compressor
20: Disinfection unit
21: Shaft
22: Connection portion
23: Disinfection member
25: Vertical transfer guide
27: Disinfecting fluid spray nozzle
30: Sensor
40: Controller
50: Sterilization device using ultraviolet light source
60: Rotation plate
R: Robot
70: Medical waste collection robot
70a: Signal transceiver
71: Collection container
72: Drive mechanism
73: Lid
74: Ultraviolet sterilizer
75: Body
76: Drive motor
76a: Drive shaft
76b: Support bearing
77: Controller
78: Drive wheel
79: Driving wheel

What is claimed is:

1. An apparatus for disinfecting a robot comprising:
a disinfection apparatus main body formed so that a robot is accommodated in an inner space thereof,
a disinfection unit installed on an inner surface of the disinfection apparatus main body to disinfect an outer surface of the robot;
a sensor formed on an inner upper surface of the disinfection apparatus main body to detect a position of the robot; and
a controller installed in the disinfection apparatus main body to control operations of the disinfection unit and the sensor.

2. The apparatus for disinfecting a robot according to claim 1,
wherein the disinfection unit comprises:
a pair of shafts coupled to connection portions extending forward from the inner surface of the disinfection apparatus main body;
a disinfection member detachably installed on the pair of shafts and configured to disinfect the outer surface of the robot;
a vertical transfer guide configured to raise and lower the connection portions;
at least one disinfecting fluid spray nozzle installed on the inner surface of the disinfection apparatus main body to spray a disinfecting fluid toward the robot or the disinfection member.

3. The apparatus for disinfecting a robot according to claim 2, further comprising:
a disinfecting fluid storage tank installed inside the disinfection apparatus main body, and configured to store and supply the disinfecting fluid to the disinfecting fluid spray nozzle; and
a disinfecting fluid pump configured to deliver the disinfecting fluid stored in the disinfecting fluid storage tank to the disinfecting fluid spray nozzle.

4. The apparatus for disinfecting a robot according to claim 2, further comprising:
a sterilization device installed on the inner upper surface or a lower surface of the disinfection apparatus main body to sterilize the robot using an ultraviolet light source.

5. The apparatus for disinfecting a robot according to claim 2, further comprising:
an air spray device installed on the inner surface of the disinfection apparatus main body to spray air toward the robot to remove contaminated particles on the surface of the robot; and
a particle collection device configured to collect the particles removed from the surface of the robot by the air spray device.

6. The apparatus for disinfecting a robot according to claim 2,
wherein, after performing disinfection on the surface of the robot as the robot in surface contact with the disinfection member rotates by itself or by a motion of the pair of shafts on which the disinfection member is installed, the controller rotates the pair of shafts to position an unused disinfection member adjacent to the robot.

7. The apparatus for disinfecting a robot according to claim 2,
wherein the controller moves the pair of shafts on which the disinfection member is installed in all directions around the robot to disinfect the outer surface of the robot.

8. The apparatus for disinfecting a robot according to claim 1, further comprising
a rotation plate installed on an inner lower surface of the disinfection apparatus main body to rotate the robot detected by the sensor about the axis of rotation in a circumferential direction.

9. The apparatus for disinfecting a robot according to claim 2, further comprising
a disinfection member collection device configured to collect the disinfection member used in the disinfection unit.

10. The apparatus for disinfecting a robot according to claim 2,
wherein the disinfecting fluid spray nozzle includes at least one spray nozzle selected from liquid type, aerosol type, and vapor type nozzles.

11. The apparatus for disinfecting a robot according to claim 1,
wherein the robot is a medical waste collection robot having a collection container configured to collect and contain medical waste, and a drive mechanism for autonomously driving the collection container, and
the apparatus for disinfecting a robot is configured as a set-type disinfection apparatus formed as one set by further including the collection robot capable of autonomously driving.

12. The apparatus for disinfecting a robot according to claim 11,
wherein the collection container of the collection robot is formed as a cylindrical container configured to contain various medical wastes, and a sterilizer which is operated by receiving a power is installed in the container.

13. The apparatus for disinfecting a robot according to claim 11,
wherein the drive mechanism that enables the collection container to autonomously drive comprises:
a body disposed below the collection container and formed to match a size and a shape of the collection container;
a drive motor installed inside the body to generate a driving force;
a controller configured to supply a power to the drive motor and control an operation thereof,
a drive wheel driven by the power received from the drive motor; and
driving wheels configured to support the body moving by the driving force of the drive wheel,
wherein the drive mechanism and the collection container are connected to a drive shaft of the drive motor through a support bearing disposed on an upper edge of the body of the drive mechanism, so that the collection container is freely rotated with respect to the drive mechanism by the driving force received from the drive motor.

14. The apparatus for disinfecting a robot according to claim 11,
wherein the medical waste collection robot comprises an electrical signal transceiver configured to transmit and receive an electrical signal to and from the sensor installed in the disinfection apparatus main body of the apparatus for disinfecting a robot.
